(19) United States
(12) Patent Application Publication
(10) Pub. No.: US 2025/0120595 A1
(43) Pub. Date: Apr. 17, 2025

(54) SYSTEMS AND METHODS TO DETERMINE AN INJURY RISK SCORE FOR A SUBJECT

(71) Applicant: GoX Studio, Inc. (dba GoX Labs), Phoenix, AZ (US)

(72) Inventors: Joseph Karl Hitt, Scottsdale, AZ (US); Robert Bruce Floersheim, Scottsdale, AZ (US)

(21) Appl. No.: 18/485,202

(22) Filed: Oct. 11, 2023

(51) Int. Cl. A61B 5/0205; A61B 5/00; A61B 5/024; A61B 5/11; G16H 50/30

(52) U.S. Cl. CPC A61B 5/02055; A61B 5/02405; A61B 5/1112; A61B 5/1116; A61B 5/6801; A61B 5/7275; A61B 5/7475; G16H 50/30; A61B 2560/0242; A61B 2562/0252; A61B 2562/0271; A61B 2562/029

(57) ABSTRACT

Systems and methods to determine an injury risk score for a subject are disclosed. Exemplary implementations may: determine, at individual instances based on output signals generated by a sensor group, parameter values for a parameter set including a load parameter, a form parameter, a fitness parameter, a fatigue parameter, and an environment parameter; obtain individual injury reports that characterize individual onsets and/or aggravations of injuries experienced by the subject during manual manipulation of one or more of the loads; determine correlations between the parameter values for the parameter set and the injury reports; determine weighted parameter values for the parameters of the parameter set based on the correlations; and determine an injury risk score for the subject by aggregating the weighted parameter values.

### FIELD OF THE DISCLOSURE

The present disclosure relates to systems and methods to determine an injury risk score for a subject.

### BACKGROUND

As a subject manually manipulates a load by exuding force with their body, multiple factor may affect muscle tissues, joints, and tendons of the subject. There fails to exist a system that quantifies the factors that may affect the body of the subject to inform the subject about their risk of injury while manipulating one or more loads.

### SUMMARY

The present disclosure is related to a system configured to determine an injury risk score for a subject by utilizing quantified factors that may affect a body of the subject while they manually manipulate a load. The factors may include a weight of the load, how the body of the subject is positioned and angled during manipulation of the load, duration and frequency in the position, how fit the subject is, how fatigued the subject is, and an ambient environment the subject is in. The subject may specify any injuries caused by or aggregated by the manual manipulation of the load. Subsequently, correlations between the factors and the injury reports may be determined. The correlations may be utilized to distinguish how significantly the individual factors each affect or cause injury to the subject. As such, the individual factors may be individually weighted and utilized in an injury risk score determination. Thus, a system that provides individualized injury risk scores determined for individual subjects is provided to improve a technical field that lacks such a system, improve existing systems that generalize the effects of various factors when determining risks for the subjects, and improve the existing systems that fail to consider all the factors when determining the risks for subjects.

One aspect of the present disclosure relates to a system configured to determine an injury risk score for a subject. The system may include a sensor group, one or more hardware processors configured by machine-readable instructions, and/or other elements. The machine-readable instructions may include one or more instruction components. The instruction components may include computer program components. The instruction components may include one or more of parameter value determination component, injury report obtaining component, correlation determination component, weight value determination component, injury risk score determination component, and/or other instruction components.

The sensor group may include of one or more sensors configured to be worn on a body of a subject. The sensor group may be further configured to generate output signal conveying information related to one or more of location of the subject, motion of the subject, form of the subject, temperature of the subject, cardiovascular parameters of the subject, and/or other information related to the subject.

The parameter value determination component may be configured to determine, at individual instances based on the output signals generated by the sensor group, parameter values for a parameter set. The parameter set may include a load parameter for individual loads that a subject manually manipulates, a form parameter for the subject, a fitness parameter for the subject, a fatigue parameter for the subject, an environment parameter for a location of the subject, and/or other parameters.

The injury report obtaining component may be configured to obtain, based on subject input via a client computing platform of the subject, individual injury reports. The individual injury reports may characterize individual onsets and/or aggravations of injuries experienced by the subject during manual manipulation of one or more of the loads.

The correlation determination component may be configured to determine correlations between the parameter values for the parameter set and the injury reports.

The weight value determination component may be configured to determine weighted parameter values for the parameters of the parameter set based on the correlations.

The injury risk score determination component may be configured to determine an injury risk score for the subject by aggregating the weighted parameter values.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof. These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1 illustrates a system configured to determine an injury risk score for a subject, the system, in accordance with one or more implementations.

FIG. 2 illustrates a method to determine an injury risk score for a subject, the system, in accordance with one or more implementations.

FIG. 3 illustrates an example implementation of the system configured to determine an injury risk score for a subject, the system, in accordance with one or more implementations.

### DETAILED DESCRIPTION

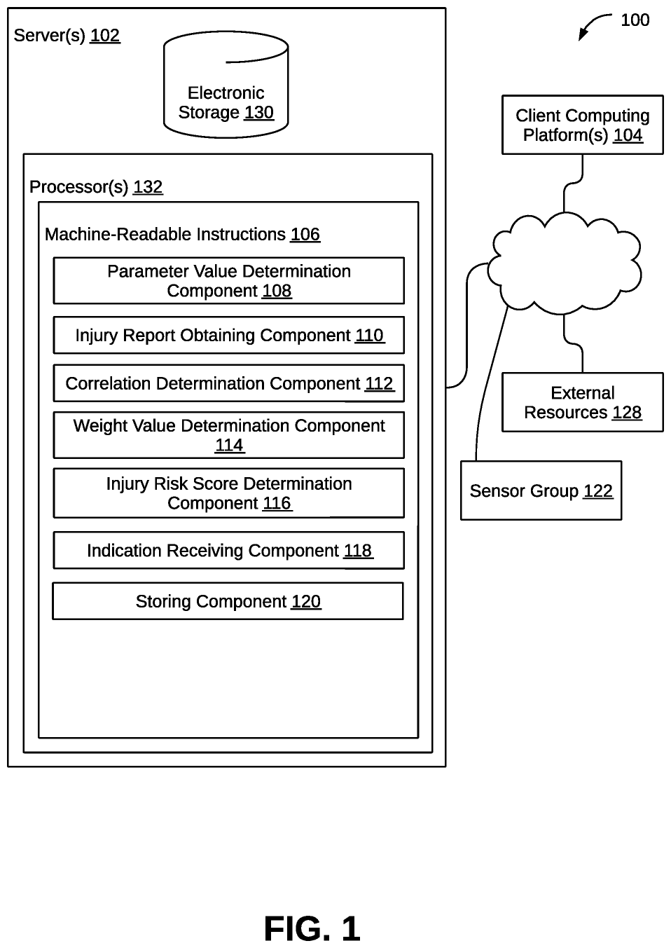

FIG. 1 illustrates a system 100 configured to determine an injury risk score for a subject, in accordance with one or more implementations. In some implementations, system 100 may include one or more servers 102, sensor group 122, and/or other elements. Server(s) 102 may be configured to communicate with one or more client computing platforms 104 and sensor group 122 according to a client/server architecture and/or other architectures. Client computing platform(s) 104 may be configured to communicate with other client computing platforms via server(s) 102 and/or according to a peer-to-peer architecture and/or other architectures. Client computing platform(s) 104 may be configured to communicate with sensor group 122 via a network. Users may access system 100 via client computing platform(s) 104.

Sensor group 122 may include of one or more sensors configured to be worn on a body of the subject. Sensor group 122 may be configured to generate output signals that convey information related to one or more of location of the subject, motion and form of the subject, a temperature of the subject, cardiovascular parameters of the subject, and/or other information about the subject. The one or more sensors may include one or more of an orientation sensor, a location sensor, a pressure sensor, a temperature sensor, a light sensor, a humidity sensor, an audio sensor, cardiovascular sensor, and/or other sensors.

The location sensor may be configured to generate output signals conveying the information related to the location of the subject and/or other information. The information related to the location derived from the output signals generated by the location sensor may define one or more of a geo-location of the subject, an elevation of the subject, and/or other measurements. A location sensor may include one or more of a GPS, an altimeter, and/or other devices. The location of the subject may be a location of a contextual environment of the subject. The contextual environment may be the immediate space surrounding the subject and of which the subject is operating and/or working in. The immediate area may be a particular radius surrounding the subject defined by the location sensor, a user, the subject, and/or by other definition. The contextual environment may change over time upon obtainment of an assigned route and/or a predicted route of the subject.

The light sensor may be configured to generate output signals conveying ambient light information and/or other information. The ambient light information derived from output signals of a light sensor may define intensity and/or presence (or absence) of light or other electromagnetic radiation incident on the light sensor. A light sensor may include one or more of a photodiode, an active-pixel sensor, photovoltaic, and/or other sensors.

The humidity sensor may be configured to generate output signals conveying humidity of the location and/or other information. The humidity sensor may include an optical sensor, gravimetric sensor, capacitive sensor, resistive sensor, piezoresistive sensor, magnetoelastic sensor, and/or other sensors.

Ambient temperature at the location of the subject may be determined based on the location of the subject, the intensity of light, the presence (or absence) of light, and/or other information conveyed by the output signals of the sensors described herein. In some implementations, the ambient temperature and humidity of the location of the subject may be obtained from external resources 128. External resources may include a weather service, for example.

The information related to the motion and form of the subject may include acceleration information, speed information, orientation information, velocity information, joint angles, duration and frequency information, and/or other information related to the motion and form of the subject. An accelerometer and/or other sensors may be configured to generate output signals conveying acceleration information of the subject. The acceleration information may specify a change in speed or direction of movement the body or part thereof. A speedometer and/or other sensors may be configured to generate output signals conveying speed information performed by the subject. The speed information may specify an amount that something occurs over a unit of time. For example, a distance ran (i.e., the motion) by the subject over a particular amount of time. The orientation sensor may be configured to generate output signals conveying the orientation information and/or other information. The orientation information derived from the output signals generated by the orientation sensor may define an orientation of the subject. The orientation may refer to one or more of a pitch angle, a roll angle, a yaw angle, heading, the direction, and/or other measurements. The orientation sensor may include an inertial measurement unit (IMU) such as one or more of an accelerometer, a gyroscope, a magnetometer, Inclinometers, Electronic nose, Infrared Imagers, Micro-bolometers, micro-displays (DMD), Digital micro-mirror device, Optical Switches, and/or other devices. The velocity information may be derived from the speed information and/or the acceleration information, and a change in the location information and/or a change in the direction. For example, the distance and the direction ran by the subject over a particular amount of time may be specified in the velocity information. The duration and frequency information may specify how long the motion performed by the subject transpired from a start to an end, how many repetitions the motion was performed by the subject during a period of time, how many repetitions the motion was performed cumulatively, and/or other duration and frequency information. The period of time may begin when the subject begins a first repetition of the motion, and end when the subject completes a final repetition of the motion. The cumulative repetitions may be a total of the repetitions of the same motion performed by the subject over multiple different periods of time.

The pressure sensor may be configured to generate output signals conveying pressure information and/or other information. Pressure information derived from output signals of a pressure sensor may define a force per unit area imparted to the pressure sensor. A pressure sensor may include one or more of a piezoresistive strain gauge, a capacitive pressure sensor, an electromagnetic pressure sensor, a piezoelectric sensor, a strain-gauge, and/or other pressure sensors.

The temperature sensor may be configured to generate output signals conveying the information related to the temperature of the subject and/or other information. The information related to the temperature of the subject derived from output signals generated by the temperature sensor may define one or more of a temperature at the temperature sensor, a temperature within a threshold range of the temperature sensor, a skin temperature reading, an internal body temperature reading, and/or other measurements of temperature of the subject. The temperature sensor may include one or more of a thermocouple, a resistive temperature Measuring device, an infrared sensor, a bimetallic device, a thermometer, and/or other temperature sensors.

An audio input sensor may be configured to receive audio input. An audio input sensor may include a sound transducer and/or other sensor configured to convert sound (e.g., air pressure variation) into an electrical signal. By way of non-limiting illustration, an audio input sensor may include a microphone.

The one or more cardiovascular sensors may be configured to generate output signals conveying biometric information related to heart, lungs, and circulation of the subject. The biometric information may include values for cardiovascular parameters of the subject, and/or other values for other biometric parameters. The cardiovascular parameters may include heart rate, a resting heart rate of the subject, a respiratory rate of the subject, blood pressure of the subject, oxygen saturation of the subject, volume of oxygen consumed (i.e., values to current and/or maximal oxygen consumption metrics), heart variability, heartbeat strength, heartbeat rhythm, and/or other cardiovascular parameters. The one or more cardiovascular sensors may include one or more an electrodermal activity (EDA), an electrocardiogramar ECG) sensor, a blood volume pulse (BVP) sensor, a respiration sensor, a blood pressure sensor, and/or other cardiovascular sensors. Other sensors that may convey the biometric information may include one or more of an electrodermal activity (EDA), an electromyography (EMG) sensor, and/or other sensors.

Server(s) 102 may be configured by machine-readable instructions 106. Machine-readable instructions 106 may include one or more instruction components. The instruction components may include computer program components. The instruction components may include one or more of parameter value determination component 108, injury report obtaining component 110, correlation determination component 112, weight value determination component 114, injury risk score determination component 116, indication receiving component 118, storing component 120, and/or other instruction components.

Parameter value determination component 108 may be configured to determine parameter values for a parameter set. The parameter values for the parameter set may be determined at individual instances based on the output signals generated by sensor group 122. The individual instances may be different occurrences at which the subject manually manipulates individual loads using one or more parts of their body. The parameter set may include a load parameter for the individual loads that the subject manually manipulates (and thus, performs motions or movements), a form parameter for the subject, a fitness parameter for the subject, a fatigue parameter for the subject, and an environment parameter for the location of the subject, and/or other parameters.

The parameter value for the load parameter may quantify a force performed by the subject to manually manipulate the individual loads. The individual loads may refer to one or more objects that the subject is collectively manipulating. For example, a load may be dumbbells, a bar bell, a package, a piece of furniture, landscaping material, among others. The subject manually manipulating the load may refer to the subject pushing, carrying, pulling, lifting over their head, squatting, curling, and/or other body movements to move the load. For example, manipulating the load may include moving an object from one location to another location (e.g., from a vehicle to a front door, from a top shelf to a bottom shelf), performing repetitions of exercises with objects of particular weights, lifting the object from a position and replacing in the same position, and/or other manual manipulations. The parameter value for the load parameter may be determined based on a load mass value of the load that is relative to a body mass of the subject. In some implementations, the load mass value of the load and/or the body mass of the subject may be input by the subject by way of subject input described herein via client computing platform 104. In some implementations, the load mass value of the load and/or the body mass of the subject may be measured via a physically or wirelessly connected scale. The relativity between the load mass value and the body mass may be conveyed in a percentage, a ratio, a fraction, a decimal, or other value format. For example, the parameter value for the load parameter may convey that the load is 15% of the subject's body mass, i.e., 15 pound load and 100 pound subject. In some implementations, the parameter value for the load parameter meeting or exceeding a load threshold may indicate a high load mass. The load threshold may be the same value format as the parameter value for the load parameter (e.g., 30%). The load threshold, and other threshold values described herein, may be fixed or modifiable by the subject, an administrative user of system 100, and/or other individuals.

The parameter value for the form parameter may quantify one or more of the orientation information, the velocity information, the acceleration information, the duration and frequency information (i.e., duration of the motion performed by the subject, frequency of the motion performed by the subject), and/or other information related to a configuration of the body during the manual manipulation of the individual loads. The orientation information may include the yaw angle, the pitch angle, and the roll angle of the a given body part. In some implementations, the orientation information may include multiple orientations of multiple different body parts. The body parts may include one or both arms, one or both elbows, one or both wrists, one or both legs, one or both feet, head, shoulders, among other body parts. For example, in a first instance, the subject may be oriented standing and their elbows bent at a 90 degree angle with fingers pointing upward towards the sky. In a second instance, the subject may be oriented laying on their back and their elbows bent at a 90 degree angle with their fingers pointing upward towards the sky. As such, the orientation of the elbows and fingers in the first instance may be different than in the second instance.

The parameter value for the fitness parameter may quantify a fitness of the subject at the individual instances of the manual manipulation of the individual loads. The fitness of the subject may correspond to a real-time state of health of the subject to perform actions. In some implementations, the real-time state of health of the subject may refer to a general state of health at a given time to function properly based on the information conveyed via sensor group 122 described herein. The fitness may consider values to a sleep metric determined based on the information conveyed by the output signals generated by the light sensor, the actimetry sensor, and/or other sensors. The values to the sleep metric may indicate sleep quality, motion during sleep, hours of sleep, and/or information related to the sleep of the subject.

The fitness may consider values to a thermal stress and dehydration metric that may indicate a risk related to thermal stress and dehydration that the subject is susceptible to. Dehydration in addition to thermal stress of the body of the subject may include an increase in core body temperature, an increase in the heart rate, an increase in oxygen consumption, a decrease in work, a decrease in efficiency of the work, a global cardiopulmonary and neural muscular chain reaction that may decrease cardiac output, stroke volume, cognitive function, and mechanical output, and/or other negative effect. The values of the thermal stress and dehydration metric may be determined by aggregating values of a water loss metric, heat index information, an exertion metric for the subject, and/or other information.

Functioning properly may include, by way of non-limiting example, the ability to stand, walk, feed oneself, and/or other everyday functions. By way of non-limiting example, actions may include walking, running, lifting, and/or other movement by the subject. The fitness of the subjects may be determined based on values to a heat index metric, the current oxygen consumption metric, the maximal oxygen consumption metric, the exertion metric, the water loss metric, the cardiovascular parameters, and/or other information as described in co-pending U.S. application Ser. No. 16/895,916 entitled “SYSTEMS AND METHODS TO DETERMINE A RISK FACTOR RELATED TO DEHYDRATION AND THERMAL STRESS OF A SUBJECT”, Attorney Docket No. 01VV-066001, the disclosure of which is incorporated by reference in its entirety herein.

In some implementations, parameter value determination component 108 may be configured to determine, based on the output signals conveying the information related the cardiovascular parameters, an average resting heart rate over a period of time. The resting heart rate may be the amount of times a heart of the subject beats per minute while at rest, i.e., not during manual manipulation of any load. The period of time may be a predefined period (e.g., one month) or may be defined by the subject or other user. Thus, over the period of time (e.g., one month), the resting heart rate of the subject may be measured multiple times and averaged to determine the average resting heart rate.

Parameter value determination component 108 may be configured to determine, based on the output signals conveying the information related the cardiovascular parameters, an average heart rate variability over the period of time. The heart rate variability may be the fluctuation of an amount of time between each heartbeat. Over the period of time (e.g., one month), the heart rate variability of the subject may be measured multiple times and averaged to determine the average heart rate variability.

In some implementations, parameter value determination component 108 may be configured to determine a first deviation of a resting heart rate before, during, and/or after individual instances from the average resting heart rate. The first deviation may refer to a difference between the resting heart rate measured at a given moment, whether before, during, and/or after individual instances, and the average resting heart rate determined.

In some implementations, parameter value determination component 108 may be configured to determine a second deviation of a heart rate variability before, during, and/or after individual instances from the average heart rate variability. The second deviation may refer to a difference between the heart rate variability measured at the given moment, whether before, during, and/or after individual instances, and the average heart rate variability determined.

In some implementations, the parameter value for the fitness parameter may be determined based on at least the first deviation of the resting heart rate and the second deviation of the heart rate variability in addition to or alternative to values to the sleep metric, the thermal stress and dehydration metric, the exertion metric, the water loss metric, the heat index metric, the current oxygen consumption metric, the maximal oxygen consumption metric, the cardiovascular parameters and/or other metrics or parameters. In some implementations, accuracy to the parameter value to the fitness parameter may be increased upon the parameter value to the fitness parameter being based on multiple values to multiple metrics and/or parameters.

The parameter value for the fatigue parameter may quantify fatigue of the subject at the instance of manual manipulation of the individual loads. In some implementations, the parameter value for the fatigue parameter may be determined based on the values of the thermal stress and dehydration metric, the sleep metric, and/or other values described herein. In some implementations, the parameter value for the fatigue parameter may be determined by dividing an oxygen volume of the subject by a maximum oxygen volume of the subject. The oxygen volume of the subject may be the volume of oxygen consumed before, during, and/or after the individual instances. The maximum oxygen volume may be a maximum volume of oxygen the subject has ever consumed. Determining the maximal oxygen volume may be from the information conveyed by the output signals. In some implementations, determining the maximum oxygen volume may be based on the information related to the subject (e.g., age, height, weight), the information related to the motion of the subject (e.g., speed of the motion), the cardiovascular information, the ambient temperature and the humidity of the location of the subject, and/or other information. Determining that a particular current oxygen consumption is the maximum oxygen volume may include comparing the particular oxygen volume with one or more of the current oxygen volume stored in electronic storage 130.

The parameter value for the environment parameter may quantify both the ambient temperature and the humidity of the location that the subject is located during the manual manipulation of the individual loads. The ambient temperature may be the average temperature of the location, which may depend on the time of year and time of day. The day of the year and the time of day may be determined based on a world clock native to client computing platform 104. The humidity may define the amount of water vapor in the air at the location.

Injury report obtaining component 110 may be configured to obtain individual injury reports. The injury reports may characterize individual onsets and/or aggravations of injuries experienced by the subject during the manual manipulation of one or more of the loads. The individual onsets of injuries may be injuries that the manual manipulation of the one or more loads may have caused or initiated. The individual aggravations of injuries may be irritations to existing injuries that the manual manipulation of the one or more loads may have caused. The individual injury reports may include one or more of the existing injuries, pain regions, a quantification of pain for the pain regions, minimized use of the pain regions, medications taken to treat the pain regions and/or injuries, therapy done to treat the pain regions and/or injuries, and/or other injury reports. The injuries may be within the pain regions. By way of non-limiting example, the injuries may include torn muscle, torn ligament, torn tendon, strained muscle, sprained ligament, strained tendon, broken bone, bruised muscle, bruised bone, concussion, blunt force trauma to one or more body parts, and/or other injuries. The pain regions may include one or both arms, one or both legs, one or both feet, one or both hips, one or both shoulders, back, abdomen, neck, and head. The pain regions may specify particular body parts, including but not limited to, elbow, knee, ankle, rib cage, shoulder blade, one or more fingers, back of head, among others.

The quantification of the pain for the pain regions may be number, percentage, letter score, or other value within a predetermined range that conveys how much pain the pain region is in. The minimized use of the pain region may characterize how the subject is avoiding or minimizing use of the pain region. By way of non-limiting example, minimized use may include limping, reduced movement or activities, lack of movement or activities, reduced pressure by using of mobility aid (e.g., wheelchair, crutches, cane), and/or other minimized uses. The medications may include one or more pain killers, muscle relaxers, and/or other medications. The medications may correspond with a dosage and frequency of use. The therapy may include use of one or more support materials (e.g., knee brace, splint, support wrap), particular exercises, particular stretches, periodic icing, chiropract therapy, and/or other therapy.

The individual injury reports may be associated with a date and time at which the subject manually manipulated the individual loads, and a date and time of the one or more onsets and/or aggravations of injuries resulting from the manual manipulation. The individual injury reports may be obtained based on subject input via client computing platform 104 of the subject. The subject input may include text input via a physical or virtual keyboard, voice input dictated via the audio sensor, selection of the information for the injury report from lists presented to the subject via a user interface of client computing platform 104, and/or other subject input.

Correlation determination component 112 may be configured to determine correlations between (a) the parameter values for the parameter set and (b) the injury reports. In some implementations, determining the correlations may include identifying one or more onsets and/or aggravations of injuries and subsequently determining outliers of the parameter values for the parameter set that were measured or determined at or near the time of the one or more onsets and/or aggravations of injuries. For example, the outliers may be particularly high parameter values for one or more of the parameters, or particularly low parameter values for one or more of the parameters. In some implementations, the individual parameters of the parameter set may be associated with a threshold value. In some implementations, the outliers may be determined based on the parameter values in relation to the individual threshold values. In some implementations, determining the correlations may include determining consistent or reoccurring parameter values to the parameter set that were measured or determined at or near the time of similar onsets and/or aggravations of injuries. In some implementations, determining the correlations may include determining similar parameter values to the parameter set that were measured or determined at or near the time of similar onsets and/or aggravations of injuries. Similar parameter values may refer to parameter values that are a deviation of ±2 concentrations, or other concentration, of the given parameter value. Such concentrations may be fixed or modifiable by the subject, the administrative user, and/or others.

In some implementations, determining multiple instances of the parameter value outliers, parameter value reoccurrences, and/or parameter value similarities related to the one or more onsets and/or aggravations of injuries may indicate a correlation. In some implementations, known or novel machine learning techniques may be employed to determine the correlations.

Weight value determination component 114 may be configured to determine individual weighted parameter values for the individual parameters of the parameter set based on the determined correlations given the measured or determined parameter values. That is, the measured or determined parameter values may be adjusted based on the correlations to determine the weighted parameter values. For example, a weighted parameter value for the form parameter may be determined based on the correlation determined between multiple parameter values for the form parameter and the injury reports, and the determined parameter value for the form parameter that is associated with a particular injury report. Determining the weighted parameter values may be include employing novel or known machine learning techniques.

In some implementations, weight value determination component 114 may be configured to determine a weight value associated with each parameter for the parameter set. The weight values may be determined based on the correlations. A given weight value associated with a given parameter of the parameter set may signify strength of the correlation between (i) a given onset and/or aggravation of injury and (ii) parameter values for the given parameter. For example, a first weight value associated with the load parameter may signify that the correlation between back injury and high load value for the load parameter (i.e., percentage of load mass relative to subject body mass is over a particular threshold) is significant. Meaning, back injuries often occur while or after manipulating high load mass relative to the body mass.

In some implementations, each of the weight values may start at a particular value (e.g., one). Based on the determined correlations, the weight values for the individual parameters may be adjusted. In some implementations, as individual ones of the correlations are enforced given the parameter values for the parameter set and the injury reports, the weight values may be further adjusted. In some implementations, determining the weight values may be based on employment of known or novel machine learning techniques.

In some implementations, weight value determination component 114 may be configured to determine weighted parameter values for the parameters (of the parameter set) based on the weight values associated with the parameters. In some implementations, the determined weight values may be multiplied by the parameter values for the respective parameters to determine the weighted parameter values for the parameters. In some implementations, the determined weight values may be added to the parameter values for the respective parameters, or other calculation, and/or other known or novel machine learning techniques to determine the weighted parameter values for the parameters.

Injury risk score determination component 116 may be configured to determine an injury risk score for the subject based on the weighted parameter values, and/or other information described herein. In some implementations, determining the injury risk score may include employing known and/or novel machine learning techniques that utilize the determined weighted parameter values. In some implementations, alternative to the parameter value to the fitness parameter that quantifies both the first deviation of the resting heart rate and the second deviation of the heart rate variability into a single value, both the first deviation of the resting heart rate and the second deviation of the heart rate variability individually may be utilized in determining the injury risk score. In some implementations, some or all the parameter values determined and/or measured as described herein and some or all of the information related to the subject manually manipulating loads determined and/or measured as described herein may be utilized as input to the known and/or novel machine learning techniques to output the injury risk score.

In some implementations, accuracy of the determined injury scores may be assessed. In some implementations, assessing the accuracy may include identifying an amount of true positive injury risk scores, an amount of false positive injury risk scores, an amount of true negative injury risk scores, and an amount of false negative injury risk scores. Subsequently, assessing the accuracy may further include dividing the amount of true positive injury risk scores by the sum of the amounts of true positive injury risk scores and false positive injury risk scores. In some implementations, the accuracy may be determined by implementing other accuracy determination techniques. In some implementations, whether the injury risk scores were true or false may be obtain via client computing platform 104 from the subjects.

In some implementations, injury risk score determination component 116 may be configured to effectuation presentation of the injury risk score via client computing platform 104 of the subject, client computing platform 104 of other users, and/or other devices. The other users may include other subjects proximate to the subject (e.g., colleagues) given their locations indicated by the output signals of the sensor groups worn by the other subjects and the subject, users not proximate to the subject (e.g., supervisors, emergency contacts), and/or other users.

In some implementations, indication receiving component 118 may be configured to receive an indication to determine the injury risk score. The indication may include a selection of a user interface element via client computing platform 104 a voice command recognized by client computing platform 104 or the wearable electronic device, a defined time reoccurrence (e.g., Monday through Friday at 10 am), and/or other indication. Responsive to receipt of the indication, the injury risk score may be determined and presented.

Storing component 120 may be configured to store the parameter values for the parameter set for the instances, the injury reports, the correlations, the weighted parameter values, the injury risk scores, and/or other information in relation to the subject and in correspondence to a time and date at which they were determined or obtained to electronic storage 130 and/or other electronic storage. By way of non-limiting example, the other electronic storage may include electronic storage on a personal device (e.g., computer, Smartphone, Smartwatch) of the subject. Such information may be stored subsequent to individual determination or obtainment.

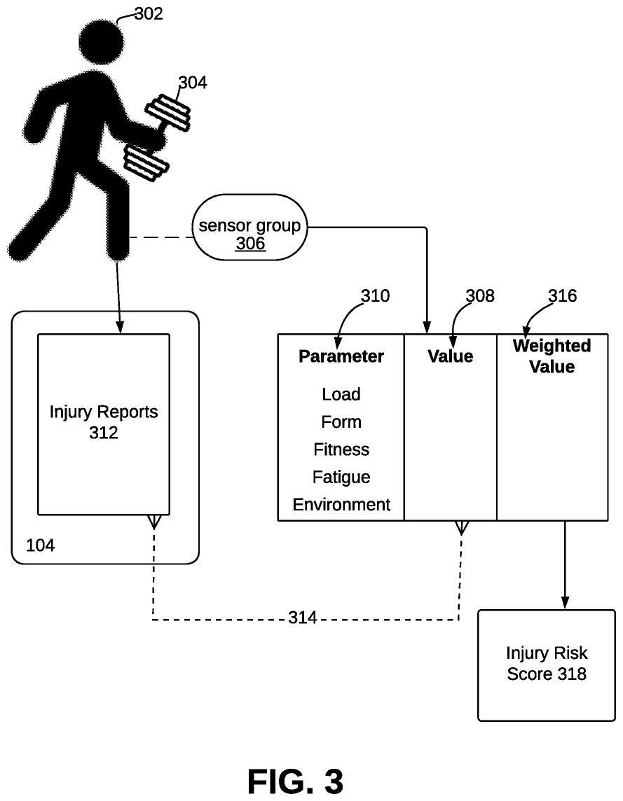

FIG. 3 illustrates a subject 302 that is manipulating a load 304. Subject 304 may be wearing or otherwise connected to sensor group 306. Based on output signals that sensor group 306 generates as subject 302 manually manipulates load 304 and other similar or different loads (not illustrated), values 308 for parameters 310 may be determined. Manual manipulation may include moving load 304 from a first point to a second point. Subject 302 may input injury reports 312 via client computing platform 104 where subject 302 may specify whether they have any onsets and/or aggravations of injuries that they experienced while manipulating load 304 and/or the other loads. Correlations 314 may be determined between values 308 and injury reports 312 to facilitate determination of weighted values 316. Based on weighted values 316, an injury risk score 318 may be determined for subject 302. Injury risk score 318 may be presented to the subject via client computing platform 104.

Referring back to FIG. 1, in some implementations, server(s) 102, client computing platform(s) 104, and/or external resources 128 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 102, client computing platform(s) 104, and/or external resources 128 may be operatively linked via some other communication media.

A given client computing platform 104 may include one or more processors configured to execute computer program components. The computer program components may be configured to enable an expert or user associated with the given client computing platform 104 to interface with system 100 and/or external resources 128, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, a wearable electronic device (e.g., Smartwatch), and/or other computing platforms.

External resources 128 may include sources of information outside of system 100, external entities participating with system 100, and/or other resources. In some implementations, some or all of the functionality attributed herein to external resources 128 may be provided by resources included in system 100.

Server(s) 102 may include electronic storage 130, one or more processors 132, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 130 may comprise non-transitory storage media that electronically stores information. The electronic storage media of electronic storage 130 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 130 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 130 may include one or more virtual storage resources (e.g., cloud storage, a virtual private network, and/or other virtual storage resources). Electronic storage 130 may store software algorithms, information determined by processor(s) 132, information received from server(s) 102, information received from client computing platform(s) 104, and/or other information that enables server(s) 102 to function as described herein.

Processor(s) 132 may be configured to provide information processing capabilities in server(s) 102. As such, processor(s) 132 may include one or more of a digital processor, an analog processor, a digital circuit designed to process information, an analog circuit designed to process information, a state machine, and/or other mechanisms for electronically processing information. Although processor(s) 132 is shown in FIG. 1 as a single entity, this is for illustrative purposes only. In some implementations, processor(s) 132 may include a plurality of processing units. These processing units may be physically located within the same device, or processor(s) 132 may represent processing functionality of a plurality of devices operating in coordination. Processor(s) 132 may be configured to execute components 108, 110, 112, 114, 116, 118, and/or 120, and/or other components. Processor(s) 132 may be configured to execute components 108, 110, 112, 114, 116, 118, and/or 120, and/or other components by software; hardware; firmware; some combination of software, hardware, and/or firmware; and/or other mechanisms for configuring processing capabilities on processor(s) 132. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 108, 110, 112, 114, 116, 118, and/or 120 are illustrated in FIG. 1 as being implemented within a single processing unit, in implementations in which processor(s) 132 includes multiple processing units, one or more of components 108, 110, 112, 114, 116, 118, and/or 120 may be implemented remotely from the other components. The description of the functionality provided by the different components 108, 110, 112, 114, 116, 118, and/or 120 described below is for illustrative purposes, and is not intended to be limiting, as any of components 108, 110, 112, 114, 116, 118, and/or 120 may provide more or less functionality than is described. For example, one or more of components 108, 110, 112, 114, 116, 118, and/or 120 may be eliminated, and some or all of its functionality may be provided by other ones of components 108, 110, 112, 114, 116, 118, and/or 120. As another example, processor(s) 132 may be configured to execute one or more additional components that may perform some or all of the functionality attributed below to one of components 108, 110, 112, 114, 116, 118, and/or 120.

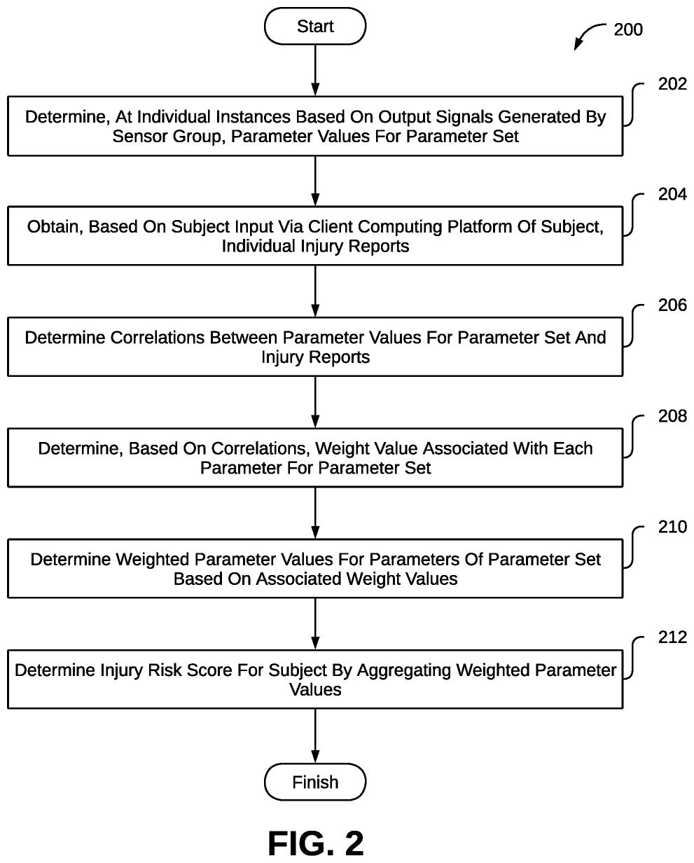

FIG. 2 illustrates a method 200 to determine an injury risk score for a subject, in accordance with one or more implementations. The operations of method 200 presented below are intended to be illustrative. In some implementations, method 200 may be accomplished with one or more additional operations not described, and/or without one or more of the operations discussed. Additionally, the order in which the operations of method 200 are illustrated in FIG. 2 and described below is not intended to be limiting.

In some implementations, method 200 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

An operation 202 may include determining, at individual instances based on output signals generated by a sensor group, parameter values for a parameter set. The parameter set may include a load parameter for individual loads that a subject manually manipulates, a form parameter for the subject, a fitness parameter for the subject, a fatigue parameter for the subject, and an environment parameter for a location of the subject. The sensor group may include of one or more sensors configured to be worn on a body of the subject. The output may signal convey information related to one or more of location of the subject, motion of the subject, form of the subject, temperature of the subject, and/or cardiovascular parameters of the subject. Operation 202 may be performed by one or more hardware processors configured by machine-readable instructions including a component that is the same as or similar to parameter value determination component 108, in accordance with one or more implementations.

An operation 204 may include obtaining, based on subject input via a client computing platform of the subject, individual injury reports that characterize individual onsets and/or aggravations of injuries experienced by the subject during manual manipulation of one or more of the loads. Operation 204 may be performed by one or more hardware processors configured by machine-readable instructions including a component that is the same as or similar to injury report obtaining component 110, in accordance with one or more implementations.

An operation 206 may include determining correlations between the parameter values for the parameter set and the injury reports. Operation 206 may be performed by one or more hardware processors configured by machine-readable instructions including a component that is the same as or similar to correlation determination component 112, in accordance with one or more implementations.

An operation 208 may include determining, based on the correlations, a weight value associated with each parameter for the parameter set. Operation 208 may be performed by one or more hardware processors configured by machine-readable instructions including a component that is the same as or similar to weight value determination component 114, in accordance with one or more implementations.

An operation 210 may include determining weighted parameter values for the parameters of the parameter set based on the associated weight values. Operation 210 may be performed by one or more hardware processors configured by machine-readable instructions including a component that is the same as or similar to weight value determination component 114, in accordance with one or more implementations.

An operation 212 may include determining an injury risk score for the subject by aggregating the weighted parameter values. Operation 212 may be performed by one or more hardware processors configured by machine-readable instructions including a component that is the same as or similar to injury risk score determination component 116, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

1. A system configured to determine an injury risk score for a subject, the system comprising:
electronic storage that stores information for the subject;
a sensor group that includes one or more sensors configured to be worn on a body of the subject, and further configured to generate output signals conveying information related to one or more of: location of the subject, motion and form of the subject, temperature of the subject, and/or cardiovascular parameters of the subject; and
one or more processors configured by machine-readable instructions to:
determine, at individual instances based on the output signals, parameter values for a parameter set including (i) a load parameter for individual loads that the subject manually manipulates, (ii) a form parameter for the subject, (iii) a fitness parameter for the subject, (iv) a fatigue parameter for the subject, and (v) an environment parameter for a location of the subject, wherein the parameter values are associated with instance times at which the output signals were generated for determination of the parameter values and stored in the electronic storage;
obtain, based on subject input via a client computing platform of the subject, individual injury reports that characterize individual onsets and/or aggravations of injuries experienced by the subject during manual manipulation of one or more of the loads, wherein the injury reports include one or more pain regions on the subject, a quantification of pain for the one or more pain regions, and one or more treatments for the one or more pain regions, wherein the injury reports are associated with report times at which the subject manually manipulated the one or more loads;
determine correlations between (a) the parameter values for the parameter set and (b) the injury reports by determining the parameter values that are temporally related to the injury reports as indicated by the report times and the instance times, and determining that the temporal relations reoccurred at least a particular amount of times;
determine weighted parameter values for the parameters of the parameter set based on the correlations;
determine the injury risk score for the subject based on the weighted parameter values; and
effectuate presentation of the injury risk score via the client computing platform.

2. The system of claim 1, wherein the one or more processors are further configured by the machine-readable instructions to receive, from the client computing platform, an indication to determine the injury risk score, wherein the injury risk score is determined in response to the indication.

3. The system of claim 1, wherein the parameter value for the load parameter is determined based on a load mass value of the load that is relative to a body mass of the subject.

4. The system of claim 1, wherein the one or more processors are further configured by the machine-readable instructions to:
determine, based on the output signals conveying information related the cardiovascular parameters, an average resting heart rate over a period of time;
determine, based on the output signals conveying information related the cardiovascular parameters, an average heart rate variability over the period of time;
determine a first deviation of a resting heart rate from the average resting heart rate;
determine a second deviation of a heart rate variability from the average heart rate variability, wherein the parameter value for the fitness parameter is determined based on at least the first deviation and the second deviation.

5. The system of claim 1, wherein the individual injury reports include minimized use of the pain regions, wherein the treatment includes medications taken and/or therapy done to treat the one or more pain regions.

6. The system of claim 1, wherein the parameter value for the load parameter quantifies a force performed by the subject to manually manipulate the individual loads.

7. The system of claim 1, wherein the parameter value for the form parameter quantifies one or more orientations and velocity of the body during manual manipulation of the individual loads.

8. The system of claim 1, wherein the parameter value for the fitness parameter quantifies a fitness of the subject at the individual instances of manual manipulation of the individual loads.

9. The system of claim 1, wherein the parameter value for the fatigue parameter quantifies fatigue of the subject at the instance of manual manipulation of the individual loads, wherein the parameter value for the fatigue parameter is determined by dividing an oxygen volume of the subject by a maximum oxygen volume of the subject.

10. The system of claim 1, wherein the parameter value for the environment parameter quantifies an ambient temperature and humidity of the location that the subject is located during manual manipulation of the individual loads.

11. A method to determine an injury risk score for a subject, the method comprising:
determining, at individual instances based on output signals generated by a sensor group, parameter values for a parameter set including (i) a load parameter for individual loads that the subject manually manipulates, (ii) a form parameter for the subject, (iii) a fitness parameter for the subject, (iv) a fatigue parameter for the subject, and (v) an environment parameter for a location of the subject, wherein the sensor group includes of one or more sensors configured to be worn on a body of the subject, wherein the output signals convey information related to one or more of: location of the subject, motion and form of the subject, temperature of the subject, and/or cardiovascular parameters of the subject, wherein the parameter values are associated with instance times at which the output signals were generated for determination of the parameter values;
obtaining, based on subject input via a client computing platform of the subject, individual injury reports that characterize individual onsets and/or aggravations of injuries experienced by the subject during manual manipulation of one or more of the loads, wherein the injury reports include one or more pain regions on the subject, a quantification of pain for the one or more pain regions, and one or more treatments for the one or more pain regions, wherein the injury reports are associated with report times at which the subject manually manipulated the one or more loads;
determining correlations between (a) the parameter values for the parameter set and (b) the injury reports by determining the parameter values that are temporally related to the injury reports as indicated by the report times and the instance times, and determining that the temporal relations reoccurred at least a particular amount of times;
determining weighted parameter values for the parameters of the parameter set based on the correlations;
determining the injury risk score for the subject based on the weighted parameter values; and
effectuating presentation of the injury risk score via the client computing platform.

12. The method of claim 11, further comprising receiving, from the client computing platform, an indication to determine the injury risk score, wherein the injury risk score is determined in response to the indication.

13. The method of claim 11, wherein the parameter value for the load parameter is determined based on a load mass value of the load that is relative to a body mass of the subject.

14. The method of claim 11, further comprising:
determining, based on the output signals conveying information related the cardiovascular parameters, an average resting heart rate over a period of time;
determining, based on the output signals conveying information related the cardiovascular parameters, an average heart rate variability over the period of time;
determining a first deviation of a resting heart rate from the average resting heart rate; and
determining a second deviation of a heart rate variability from the average heart rate variability, wherein the parameter value for the fitness parameter is determined based on at least the first deviation and the second deviation.

15. The method of claim 11, wherein the individual injury reports include minimized use of the pain regions, wherein the treatment includes medications taken and/or therapy done to treat the one or more pain regions.

16. The method of claim 11, wherein the parameter value for the load parameter quantifies a force performed by the subject to manually manipulate the individual loads.

17. The method of claim 11, wherein the parameter value for the form parameter quantifies one or more orientations and velocity of the body during manual manipulation of the individual loads.

18. The method of claim 11, wherein the parameter value for the fitness parameter quantifies a fitness of the subject at the individual instances of manual manipulation of the individual loads.

19. The method of claim 11, wherein the parameter value for the fatigue parameter quantifies fatigue of the subject at the instance of manual manipulation of the individual loads, wherein the parameter value for the fatigue parameter is determined by dividing an oxygen volume of the subject by a maximum oxygen volume of the subject.

20. The method of claim 11, wherein the parameter value for the environment parameter quantifies an ambient temperature and humidity of the location that the subject is located during manual manipulation of the individual loads.
